(19) United States
(12) Patent Application Publication
(10) Pub. No.: US 2015/0059794 A1
(43) Pub. Date: Mar. 5, 2015

(54) PROCESS FOR STRAIGHTENING THE HAIR

(71) Applicant: KAO CORPORATION, Tokyo (JP)

(72) Inventors: Burkard ROSE, Darmstadt (DE); Jonathan Wood, Weinheim (DE)

(21) Appl. No.: 14/470,111

(22) Filed: Aug. 27, 2014

Related U.S. Application Data
(60) Provisional application No. 61/872,002, filed on Aug. 30, 2013.

(51) Int. Cl. A61K 8/33; A45D 2/00; A61K 8/40; A45D 7/06; A61Q 5/10; A61Q 5/04

(52) U.S. Cl. CPC A61K 8/33; A61Q 5/10; A61Q 5/04; A61K 8/40; A45D 7/06; A45D 2/001; A61K 2800/4324; A45D 2007/001
USPC 132/206; 8/405; 424/70.2; 132/286

(57) ABSTRACT

The present invention provides methods for simultaneously colouring and straightening the hair, for straightening the hair directly before or after a colouring treatment, and for straightening the hair directly before or after a bleaching treatment. Besides, the present invention also provides a method for straightening the hair by drying the hair with hot air under conditions of mechanical tension. The straightening methods of the present invention utilize compositions containing formaldehyde.

### RELATED APPLICATIONS

The present application claims priority to U.S. provisional application No. 61/872,002 filed Aug. 30, 2013, which is incorporated herein by reference in its entirety.

### FIELD OF THE INVENTION

The present invention relates to a method for one step colouring and semipermanent straightening of the hair.

### BACKGROUND OF THE INVENTION

A known method for straightening curly or frizzy hair involves the use of straightening irons. The high temperature of the iron leads to a breakage of hydrogen bonds in the keratin of the hair, achieving a temporary straightening. The hydrogen bonds are formed again by the action of moisture, so that the hair reverts back to its original shape over the time because of air humidity, and the straightening effect vanishes after washing the hair.

The shape of the hair is largely determined by the disulfide bonds linking two cysteine moieties of the hair keratin. In order to achieve a more permanent shaping of the hair, known methods involve the cleavage of the disulfide bonds by the action of a sulfide- or thio group containing reducing agent. After the hair has been brought into the desired shape, new disulfide bonds are formed by applying an oxidizing agent such as hydrogen peroxide, thus fixing the shape of the hair. The use of such agents, however, may cause damage to the hair.

As an example for this kind of hair shaping treatment, reference is made to GB 1 416 564, describing reducing compositions comprising thioglycolates or thiolactates as reducing agents and fixing compositions comprising hydrogen peroxide as an oxidizing agent. The reducing compositions may further comprise a salt of an acid such as glyoxylic acid as a buffering agent.

As an alternative to the above-described two-step reduction and oxidation process, the disulfide bridges can be cleaved by the action of an alkaline agent such as sodium hydroxide at a pH of about 11 or higher. Under these conditions, the disulfide (or cystin) moiety can undergo a disproportionation reaction under the elimination of sulfur, and is cleaved into an alpha-beta-unsaturated dehydro-alanine moiety and a cysteine moiety. After the hair has been brought into the desired shape, the dehydro-alanin moieties and the cysteine moieties form thioether bonds and combine to lanthionine, stabilizing the straightened state of the hair. Since the disulfide or cystin moieties are converted into lanthionine moieties, this type of hair straightening process using an alkaline agent is also called lanthionization.

Both the two-stage reduction/oxidation method and the lanthionization method rely on a cleavage of the disulfide bonds and the formation of new bonds among the hair proteins, leading to an irreversible change of the shape of the hair. This means that these processes can achieve a permanent straightening, wherein the treated portion of the hair maintains its shape, and the straightening effect only vanishes because of the growth of the hair.

Recently, it has been found out that formaldehyde comprising compositions effectively straighten hair when used together with a thermal/mechanical straightening means. Such methods are described in US 2006/104928, and by A. Valdez in Cosmetiscope, Edition of Summer 2011 (Vol. 17 No. 6) (available online under www.nyscc.org).

Hair dyeing methods and agents may be categorized in accordance with the type of the dye and the permanency of the color on hair. Depending on the permanency of the color, hair dyes are usually classified as “permanent”, “demi-permanent”, “semi-permanent” or “temporary”.

Permanent and demi-permanent dyeing is typically performed with oxidation dyes, which are formed from low molecular intermediates known as “precursors” and “couplers” by oxidative coupling thereof. The intermediates are small enough to penetrate into the hair, while after the application of the oxidizing agent, the larger dye molecule formed by the oxidative coupling stays trapped inside the hair.

Conversely, in case of direct dyes, the actual dye molecule is applied to the hair directly and adheres to the hair surface because of, for example, electrostatic interactions. In contrast to the oxidation dye intermediates, the penetration of the direct dyes into the hair is relatively poor because of the larger molecule size. As a result, direct dyes can be washed out, so that the coloring is merely temporary.

Some direct dyes, in particular nitro dyes, may adhere more firmly to the hair and may penetrate deeper into the hair surface to some extent. Coloring compositions comprising these dyes are washed out less easily and thus are termed “semi-permanent”.

Hair bleaching involves the oxidative decomposition of the melanin pigments in the hair by the action of an oxidizing agent, thus bleaching, blonding or lightening the hair colour. Typically, alkaline hydrogen peroxide or peroxide compounds such as persulfates are used as the oxidizing agent.

The hair bleaching composition is generally of a two-component type, wherein the first component is a water-free composition comprising an alkalizing agent, while the second component is an aqueous hydrogen peroxide solution. The components are mixed prior to the application to the hair.

The water-free first component may be a bleaching powder comprising a persulfate in combination with an alkaline agent. In order to reduce the dustiness, the powder may be agglomerated with a binding agent, yielding a granular composition. A usual binding agent is mineral oil, which is the subject matter of EP 560 088 B1. Furthermore, EP 778 020 A1 suggests the use of oil and wax compounds or their mixtures for preparation of suspensions.

Because of the strong oxidative activity, the hair may be damaged in the bleaching process, specifically in case the hair is repeatedly bleached. EP 2 468 247 addresses the problem of alleviating the risk of hair damage and describes a substantially water-free hair bleaching and/or lightening composition characterized by comprising a diamide compound.

### SUMMARY OF THE INVENTION

The present invention relates to hair straightening methods and the combination of hair straightening and hair dyeing methods or bleaching methods. The aspects of the present invention may be summarized as follows:
i—The hair is simultaneously dyed and straightened with a dyeing/straightening composition used in combination with a straightening iron;
ii—The hair is dyed first with a dye Composition and straightened afterwards with a straightening composition used in combination with a straightening iron;
iii—The hair is straightened first with a straightening composition used in combination with a straightening iron and afterwards dyed with a dye composition;
iv—The hair is bleached first with a bleaching composition and straightened afterwards with a straightening composition used in with a straightening iron;
v—The hair is straightened first with a straightening composition used in combination with a straightening iron and afterwards bleached with a bleaching composition;
vi—The hair is straightened with a straightening composition used in combination with a hot air stream while the hair is held under mechanical tension;

In the above aspects i to vi of the present invention, the straightening composition and the dyeing/straightening composition both contain formaldehyde as the active agent for providing the straightening effect. The dyeing/straightening composition further contains at least one direct dye.

The dye composition used in the above aspects ii and iii contains at least one hair dye, such as at least one direct dye, at least one oxidation dye or a combination thereof. In case the composition contains an oxidation dye and/or if it is desired to additionally provide a bleaching or lightening effect, then an oxidizing agent such as hydrogen peroxide may be added. Typically, the oxidizing agent is stored separately and added to the dye composition immediately before application.

The bleaching composition used in the above aspects iv an v contains at least one oxidizing agent such as hydrogen peroxide and at least one alkalizing agent. Typically, the oxidizing agent and the alkalizing agent are stored separately and mixed immediately before application.

In the aspects i to v of the present invention, after the dyeing/straightening composition or the straightening composition has been applied to the hair, the hair is straightened with an iron having a surface temperature of 130 to 250° C. Alternatively, in aspect vi of the present invention, the hair may be held under mechanical tension while applying a hot air flow having a temperature of 60 to 140° C.

### DESCRIPTION OF SPECIFIC EMBODIMENTS

Accordingly, an embodiment of the first aspect of the present invention is directed to a process for simultaneously dyeing and straightening the hair, comprising the following steps performed in this order:
(i-a) application of a dyeing/straightening composition comprising at least one direct dye and formaldehyde onto the hair;
(i-b) leaving the composition on the hair for 1 to 120 minutes;
(i-c) optionally rinsing off the hair;
(i-d) drying the hair;
(i-e) treating the hair with an iron having a surface temperature of 130 to 250° C.; and
(i-f) optionally rinsing off and/or shampooing the hair and drying.

An embodiment of the second aspect of the present invention relates to a process for dyeing and straightening the hair, comprising the following steps performed in this order:
(ii-a) application of a first composition (dye composition) comprising at least one hair dye, optionally at least one alkalizing agent and optionally at least one oxidizing agent onto the hair,
(ii-b) leaving the composition on the hair for 5 to 45 minutes, rinsing off the composition and optionally shampooing the hair,
(ii-c) optionally drying the hair,
(ii-d) application of a second composition (straightening composition) comprising formaldehyde,
(ii-e) leaving the composition on the hair for 1 to 120 minutes,
(ii-f) drying the hair,
(ii-g) optionally rinsing off the hair with water and drying the hair,
(ii-h) treating the hair with an iron having a surface temperature of 130 to 250° C., and
(ii-i) optionally rinsing off the hair with water and drying the hair,
   wherein the steps (ii-d) to (ii-i) are carried out directly after steps (ii-a) to (ii-c).

As regards the third aspect of the present invention, one embodiment thereof relates to a process for straightening and dyeing the hair, comprising the following steps performed in this order:
(iii-a) application of a first composition (straightening composition) comprising formaldehyde,
(iii-b) leaving the composition on the hair for 1 to 120 minutes,
(iii-c) drying the hair
(iii-d) optionally rinsing off the hair with water and drying the hair,
(iii-e) treating the hair with an iron having a surface temperature of 130 to 250° C.,
(iii-f) optionally rinsing and/or shampooing and drying the hair,
(iii-g) application of a second composition (dye composition) comprising at least one hair dye, optionally at least one alkalizing agent and optionally at least one oxidizing agent onto the hair,
(iii-h) leaving the composition on the hair for 5 to 45 minutes, rinsing off the composition and optionally shampooing and drying the hair,
   wherein steps (iii-g) to (iii-h) are carried out directly after steps (iii-a) to (iii-f).

An embodiment of the fourth aspect of the present invention concerns a process for bleaching and straightening hair, which comprises the following steps performed in this order:
(iv-a) Application of an aqueous first composition (bleaching composition) comprising at least one oxidizing agent and at least one alkalizing agent onto the hair,
(iv-b) leaving the composition on the hair for up to 45 minutes, rinsing off the composition and optionally shampooing the hair,
(iv-c) optionally drying the hair,
(iv-d) application of a second composition (straightening composition) comprising formaldehyde,
(iv-e) leaving the composition on the hair for 1 to 120 minutes;
(iv-f) drying the hair,
(iv-g) treating the hair with an iron having a surface temperature of 130 to 250° C.
   wherein steps (iv-d) to (iv-g) are carried out directly after steps (iv-a) to (iv-c).

Turning to the fifth aspect of the present invention, an embodiment thereof concerns a process for straightening and bleaching hair, which comprises the following steps performed in this order:
(v-a) application of a first composition (straightening composition) comprising formaldehyde,
(v-b) leaving the composition on the hair, for 1 to 120 minutes;
(v-b′) optionally rinsing of the hair;
(v-c) drying the hair,
(v-d) treating the hair with an iron having a surface temperature of 180+/−50° C.,
(v-e) rinsing the hair and optionally shampooing and drying the hair
(v-f) application of an aqueous second composition (bleaching composition) comprising at least one oxidizing agent and at least one alkalizing agent onto the hair,
(v-g) leaving the composition on the hair for 5 to 45 minutes, rinsing off the composition and optionally shampooing the hair,
   wherein steps (v-e) to (v-g) are carried out directly after steps (v-a) to (v-d).

An embodiment of the sixth aspect of the present invention relates to a process for straightening hair, comprising the following steps performed in this order:
(vi-a) application of a hair straightening composition comprising formaldehyde,
(vi-b) subsequently leaving the composition on the hair for 1 to 45 minutes;
(vi-c) optionally rinsing off the hair;
(vi-d) drying the hair under conditions of mechanical tension and hot air flow having a temperature of 60-140° C.; and
(vi-e) optionally rinsing off and/or shampooing the hair.

### DETAILED DESCRIPTION OF THE INVENTION

Conventionally, coloring and straightening treatment of the hair is carried out in two separate steps, which is time consuming and uneconomical for end users. The first aspect of the present invention solves this problem by providing a method which achieves a simultaneous coloring and straightening of the hair in a single step.

For this purpose, the method of the present invention uses a hair treatment composition comprising formaldehyde and at least one direct dye.

Surprisingly, it has been found that the formaldehyde and the dye can be applied to the hair simultaneously without adverse interactions. On the contrary, the use of the hair treatment composition in accordance with the present invention unexpectedly improves durability of colors obtained with direct dye comprising compositions.

Other aspects of the present invention concern the coloring and straightening of the hair in two successive steps, the bleaching and straightening of the hair in two successive steps and the straightening of the hair by drying the hair under mechanical tension.

### 1. The Dyeing/Straightening Composition and the Straightening Composition

The dyeing/straightening composition of the first aspect of the present invention and the straightening composition of the second to sixth aspect comprise formaldehyde. The dyeing/straightening composition further contains at least one direct dye.

Besides, the dyeing/straightening composition and the straightening composition may also comprise at least one surfactant and/or at least one conditioning component. Appropriate examples and amounts thereof are explained in detail below. For promoting the ease of use, it is particularly preferable to include a silicone, preferably an amodimethicone, and/or a cationic polymer into the composition.

The dyeing/straightening composition and the straightening composition may suitably be in the form of a solution, emulsion, cream, gel, paste and mousse. When formulating the composition as a gel, a polymeric thickening agent may be added, as described below. Preferably, the composition is formulated as an aqueous solution.

The pH of the dyeing/straightening composition and the straightening composition is in the range of 1 to 6, preferably 1.5 to 5.5, more preferably 2 to 5 and most preferably 3 to 5.

The dyeing/straightening composition and the straightening composition may be formulated as a one-part composition, or as a two-part composition comprising the parts A and B, which are stored separately and mixed prior to the application to the hair. For instance, part A may comprise the formaldehyde and part B may comprises at least one of a fragrance, a surfactant or a conditioning component and, in case of the dyeing/straightening composition, at least one direct dye.

### 2. Formaldehyde

The dyeing/straightening composition and the straightening composition used in the present invention comprise formaldehyde (HCHO). When formulated as an aqueous solution, the formaldehyde may be present in the form of its hydrate H₂C(OH)₂ or in an oligomeric or polymeric form.

In the composition, the formaldehyde is preferably present in a concentration of 0.1 to 20 wt. %, more preferably 1 to 15 wt. %, even more preferably 2 to 12.5 wt. % in terms of HCHO, based on the total weight of the composition.

As discussed above, conventional permanent hair shaping/straightening techniques are based on the re-organization of the disulfide bridges and involve a cleavage of the disulfide bonds either by using a sulfur-based reducing agent, followed by the shaping of the hair and the formation of new bonds (i.e., disulfide bonds formed by the action of an oxidizing agent or thioether bonds, respectively). In contrast to these permanent straightening methods, the present invention does not utilize cleavage of the disulfide bonds and fixing the bonds in the new shape. Therefore, the dyeing/straightening composition and the straightening composition of the present invention do not require the presence of sulfur-based reducing agents. However, up to 2% by weight calculated to the total of the composition sulfur based reducing agents does not disturb the straightening performance of the compositions.

### 3. The Direct Dye

In the dyeing/straightening composition of the first aspect of the present invention, there are no particular limitations as to the type of direct dye, and any direct dye suitable for hair coloring may be used. Examples of the direct dye include an anionic dye, a nitro dye, a disperse dye, and a cationic dye and mixtures thereof.

Non-limiting examples of the cationic dyes are Basic Blue 6, Basic Blue 7, Basic Blue 9, Basic Blue 26, Basic Blue 41, Basic Blue 99, Basic Brown 4, Basic Brown 16, Basic Brown 17, Natural Brown 7, Basic Green 1, Basic Orange 31, Basic Red 2, Basic Red 12 Basic Red 22, Basic Red 51, Basic Red 76, Basic Violet 1, Basic Violet 2, Basic Violet 3, Basic Violet 10, Basic Violet 14, Basic Yellow 57 and Basic Yellow 87 and mixtures thereof. Particularly preferred are Basic Red 51, Basic Orange 31, Basic Yellow 87 and mixtures thereof.

Non-limiting examples of the anionic dyes are Acid Black 1, Acid Blue 1, Acid Blue 3, Food Blue 5, Acid Blue 7, Acid Blue 9, Acid Blue 74, Acid Orange 3, Acid Orange 6, Acid Orange 7, Acid Orange 10, Acid Red 1, Acid Red 14, Acid Red 18, Acid Red 27, Acid Red 50, Acid Red 52, Acid Red 73, Acid Red 87, Acid Red 88, Acid Red 92, Acid Red 155, Acid Red 180, Acid Violet 9, Acid Violet 43, Acid Violet 49, Acid Yellow 1, Acid Yellow 23, Acid Yellow 3, Food Yellow No. 8, D&C Brown No. 1, D&C Green No. 5, D&C Green No. 8, D&C Orange No. 4, D&C Orange No. 10, D&C Orange No. 11, D&C Red No. 21, D&C Red No. 27, D&C Red No. 33, D&C Violet 2, D&C Yellow No. 7, D&C Yellow No. 8, D&C Yellow No. 10, FD&C Red 2, FD&C Red 40, FD&C Red No. 4, FD&C Yellow No. 6, FD&C Blue 1, Food Black 1, Food Black 2, Disperse Black 9 and Disperse Violet 1 and their alkali metal salts such as the sodium or potassium salt and mixtures thereof.

Among those, the preferred anionic dyes are Acid Red 52, Acid Violet 2, Acid Red 33, Acid Orange 4, Acid Red 27 and Acid Yellow 10 and their salts. The most preferred anionic dyes are Acid Red 52, Acid Violet 2, Acid Red 33, Acid Orange 4 and Acid Yellow 10, and their salts and mixtures thereof.

Non-limiting examples for the nitro dye are HC Blue No. 2, HC Blue No. 4, HC Blue No. 5, HC Blue No. 6, HC Blue No. 7, HC Blue No. 8, HC Blue No. 9, HC Blue No. 10, HC Blue No. 11, HC Blue No. 12, HC Blue No. 13, HC Brown No. 1, HC Brown No. 2, HC Green No. 1, HC Orange No. 1, HC Orange No. 2, HC Orange No. 3, HC Orange No. 5, HC Red BN, HC Red No. 1, HC Red No. 3, HC Red No. 7, HC Red No. 8, HC Red No. 9, HC Red No. 10, HC Red No. 11, HC Red No. 13, HC Red No. 54, HC Red No. 14, HC Violet BS, HC Violet No. 1, HC Violet No. 2, HC Yellow No. 2, HC Yellow No. 4, HC Yellow No. 5, HC Yellow No. 6, HC Yellow No. 7, HC Yellow No. 8, HC Yellow No. 9, HC Yellow No. 10, HC Yellow No. 11, HC Yellow No. 12, HC Yellow No. 13, HC Yellow No. 14, HC Yellow No. 15, 2-Amino-6-chloro-4-nitrophenol, picramic acid, 1,2-Diamino-4-nitrobenzol, 1,4-Diamino-2-nitrobenzol, 3-Nitro-4-aminophenol, 1-Hydroxy-2-amino-3-nitrobenzol and 2-hydroxyethylpicramic acid and mixtures thereof.

The dyeing/straightening composition may suitably comprise only one direct dye or a combination of direct dyes. In this respect, direct dyes of different ionic characters may also be comprised in the same composition. The total content of direct dye in the treatment composition is within the range of 0.001 to 10%, preferably 0.01 to 7.5%, more preferably 0.05 to 5% by weight, based on the total weight of the dyeing/straightening composition.

### The Dye Composition

The dye composition used in the second and third aspect of the present invention is not particularly limited and may be a direct dye composition or an oxidation dye composition. For instance, the oxidation dye compositions as described in EP 2 407 149 or the compositions as described in EP 2 329 809, which comprise a combination of an oxidation dye and a direct dye may be used.

The dye composition may suitably comprise further ingredients such as surfactants and/or conditioning agents as defined below, and may suitably be in the form of a solution, emulsion, cream, gel, paste and mousse.

### Direct Dye

The dye composition may comprise a direct dye. The same direct dyes as described above for the dyeing/straightening composition may be used.

The dye composition may suitably comprise only one direct dye or a combination of direct dyes. In this respect the direct dyes of different ionic characters may also be comprised in the same composition.

The total amount of direct dye in the composition is within the range of 0.001 to 10%, preferably 0.01 to 7.5%, more preferably 0.05 to 5% by weight, based on the total weight of the dye composition.

If the dye composition only comprises direct dyes, an oxidizing agent is not required for coloring the hair. In case lightening of the hair color is desired, an oxidizing agent may be included into the composition.

### Oxidation Dye

In case the dye composition comprises an oxidation dye, it is typically of a two-component type, wherein the first component comprises the oxidation dye intermediates (precursor and coupler) and an alkalizing agent, while the second component comprises an oxidizing agent such as hydrogen peroxide. The two components are typically stored separately and combined before application onto the hair.

In the present invention, there are no particular limitations as to the oxidation dye intermediates, any known precursors and couplers normally used in a hair dye product may suitably be used.

Non-limiting examples of the precursor include para-phenylenediamine, toluene-2,5-diamine, 2-chloro-para-phenylenediamine, N-methoxyethyl-para-phenylenediamine, N,N-bis(2-hydroxyethyl)-para-phenylenediamine, 2-(2-hydroxyethyl)-para-phenylenediamine, 2,6-dimethyl-para-phenylenediamine, 4,4′-diaminodiphenylamine, 1,3-bis(N-(2-hydroxyethyl)-N-(4-aminophenyl)amino)-2-propanol, PEG-3,3,2′-para-phenylenediamine, para-aminophenol, para-methylaminophenol, 3-methyl-4-aminophenol, 2-aminomethyl-4-aminophenol, 2-(2-hydroxyethylaminomethyl)-4-aminophenol, ortho-aminophenol, 2-amino-5-methylphenol, 2-amino-6-methylphenol, 2-amino-5-acetamide phenol, 3,4-diaminobenzoic acid, 5-aminosalicylic acid, 2,4,5,6-tetraaminopyrimidine, 2,5,6-triamino-4-hydroxypyrimidine, 4,5-diamino-1-(4′-chlorobenzyl)pyrazole, 4,5-diamino-1-hydroxyethylpyrazole, and salts of these substances and their mixtures.

Non-limiting examples of the coupler include meta-phenylenediamine, 2,4-diaminophenaxyethanol, 2-amino-4-(2-hydroxyethylamino)anisole, 2,4-diamino-5-methylphenetole, 2,4-diamino-5-(2-hydroxyethoxyl)toluene, 2,4-dimethoxy-1,3-diaminobenzene, 2,6-bis(2-hydroxyethylamino)toluene, 2,4-diamino-5-fluorotoluene, 1,3-bis(2,4-diaminophenoxy)propane, meta-aminophenol, 2-methyl-5-aminophenol, 2-methyl-5-(2-hydraxyethylamino)phenol, 2,4-dichloro-3-aminophenol, 2-chloro-3-amino-6-methylphenol, 2-methyl-4-chloro-5-aminophenol, N-cyclopentyl-meta-aminophenol, 2-methyl-4-methoxy-5-(2-hydroxyethylamino)phenol, 2-methyl-4-fluoro-5-aminophenol, resorcin, 2-methylresorcin, 4-chlororesorcin, 1-naphthol, 1,5-dihydroxynaphthalene, 1,7-dihydroxynaphthalene, 2,7-dihydroxynaphthalene, 2-isopropyl-5-methylphenol, 4-hydroxyindole, 5-hydroxyindole, 6-hydroxyindole, 7-hydroxyindole, 6-hydroxybenzomorpholine, 3,4-methylenedioxyphenol, 2-bromo-4,5-methylenedioxyphenol, 3,4-methylenedioxyaniline, 1-(2-hydroxyethyl)amino-3,4-methylenedioxybenzene, 2,6-dihydroxy-3,4-dimethylpyridine, 2,6-dimethoxy-3,5-diaminopyridine, 2,3-diamino-6-methoxypyridine, 2-methylamino-3-amino-6-methoxypyridine, 2-amino-3-hydroxypyridine, 2,6-diaminopyridine, and salts of these substances and their mixtures.

The concentration of the dye precursors and couplers each is in the range of 0.001 to 10%, preferably 0.01 to 7.5%, more preferably 0.1 to 5% by weight based on the total weight of the dye composition.

The oxidation dye composition further comprises an alkalizing agent. Suitable non-limiting examples of the alkalizing agent include ammonia and a salt thereof; alkanolamine such as monoethanolamine, isopropanolamine, 2-amino-2-methyl propanol, and 2-aminobutanol, and a salt thereof; alkanediamine such as 1,3-propanediamine and a salt thereof; and carbonate such as guanidine carbonate, sodium carbonate, potassium carbonate, sodium bicarbonate, and potassium bicarbonate and their mixtures. The concentration of the alkalizing agent is in the range of 0.1 to 15% preferably 0.5 to 10%, more preferably 1 to 7.5% by weight, based on the total weight of the dye composition.

In the oxidation dye composition, the component comprising the oxidizing agent is stored separately and mixed with the dye component prior to application onto hair. Suitable non-limiting examples of the oxidizing agent include hydrogen peroxide, urea peroxide, melamin peroxide and sodium bromate. Among these oxidizing agents, hydrogen peroxide is preferable.

The concentration of the at least one oxidizing agent, preferably hydrogen peroxide, is in the range of 1 to 20%, preferably 1 to 15%, more preferably 1 to 12% and even more preferably 2 to 9% by weight, based on the total weight of the dye composition.

When stored separately, the pH of the component comprising the oxidizing agent is preferably 2 to 6, more preferably 2.5 to 4. The pH may be adjusted by means of suitable buffering agents.

After mixing the dye component with the oxidizing component, the pH of the dye composition is typically in the range of 6 to 11, preferably 6.5 to 10.5 and more preferably 6.8 to 10, measured at ambient temperature (25° C.)

The oxidation dye composition may additionally comprise at least one direct dye. The direct dyes mentioned above are all suitable for this purpose.

### 5. The Bleaching Composition

The bleaching, blonding or lightening of the hair colour is achieved by applying a bleaching composition which causes the oxidative decomposition of the melanin pigments in the hair. The bleaching composition used in the fourth and fifth aspect of the present invention comprises at least one oxidizing agent and at least one alkalizing agent. Typically, alkaline hydrogen peroxide or peroxide compounds such as persulfates are used as the oxidizing agent in an aqueous medium.

There are no particular limitations with respect to the bleaching composition used in the process of the present invention. Any conventional bleaching, blonding or lightening composition may be used.

The concentration of hydrogen peroxide in the bleaching composition is usually within the range of 1 to 20 wt. %, preferably 1 to 15 wt. %, more preferably 2 to 12 wt. %, based on the total weight of the composition, depending on the desired degree of bleaching. In order to increase the bleaching effect, hydrogen peroxide and other peroxide compounds may be used in combination. The bleaching composition has an alkaline pH of usually from 8 to 11, in particular 9 to 10.

Typically, the bleaching composition comprises at least two components, which are stored separately and mixed prior to application onto the hair. The first component comprises an alkalizing agent and the second component is an aqueous hydrogen peroxide composition, wherein the two components are stored separately and mixed prior to application onto the hair. Examples of the alkalizing agent in the first component include ammonia, mono ethanol amine, isopropanolamine, 2-amino-2-methyl propanol, 2-amino-2-hydroxy-1,3-propanediol and 2-aminobutanol, and a salt thereof; alkanediamine such as 1,3-propanediamine and a salt thereof; and carbonate such as guanidine carbonate, sodium carbonate, potassium carbonate, sodium bicarbonate, and potassium bicarbonate and their mixtures.

The aqueous hydrogen peroxide solution preferably has a concentration of 1 to 20 wt. %, more preferably 2 to 12 wt. %, based on the total weight of the composition. The pH is preferably 2 to 6, more preferably 2.5 to 4. The pH may be adjusted by means of suitable organic and inorganic acids, bases and buffering agents.

The hydrogen peroxide of the second component may be used as the only oxidizing agent. Bleaching compositions of this type are usually termed “lightening compositions”. It is possible to use a mixture of an aqueous ammonia solution and an aqueous hydrogen peroxide solution as a lightening composition.

As a preferable example for a lightening composition, an aqueous solution of 3-5 wt. % ammonia and 3-5 wt. % hydrogen peroxide may be mentioned. The aqueous solutions of ammonia and the hydrogen peroxide are mixed directly before use.

For increasing the bleaching effect, however, the first component preferably contains a further oxidizing agent in solid form. As examples for the oxidizing agent, persulfates such as sodium, potassium and ammonium persulfate, earth alkali peroxides such as magnesium peroxide, melamine peroxide or urea peroxide or phthalimide peroxyhexanoic acid, and mixtures thereof may be mentioned. The proportion of the oxidizing agent in the first component is at least 5%, preferably in the range of 20 to 80%, more preferably 25 to 70% and most preferably 30 to 60% by weight, based on the total weight of the composition. Bleach powders, such as those described in EP 2 468 247, may be mentioned as examples for this type of compositions.

For a bleaching composition with a stronger bleaching effect, it is preferable to use a bleach powder comprising a persulfate salt such as sodium, potassium and ammonium persulfate, in an amount of 20 to 60 wt. % and an alkali metasilicate such as sodium silicate as a basic component in an amount of 5 to 15 wt. %, based on the total weight of the bleaching powder. The remainder may be a powder base material such as diatomeous earth. The bleach powder is mixed with 4 to 12 wt. % hydrogen peroxide composition at a ratio of preferably 1:1 before use.

### 6. Surfactant

Any of the above-described dyeing/straightening compositions the straightening compositions, the dye compositions and the bleaching compositions (in the following collectively referred to as “treatment compositions”) may comprise a surfactant. As the surfactant, any of a cationic surfactant, a nonionic surfactant, an amphoteric surfactant and an anionic surfactant can be used. It is also possible to use two or more types of surfactants in combination.

The cationic surfactant is selected from a mono-long chain alkyl quaternary ammonium salt, having a C₈-C₂₄ alkyl residue and three C₁-C₄ alkyl residues and quaternary ammonium salts having two C₅-C₂₄ linear or branched, saturated or unsaturated hydrocarbon groups in the molecule, which may be the same or different and may be optionally substituted with one or more substituents selected from halogen, hydroxyl, amino and C₁-C₄ alkoxy.

Mono alkyl quaternary ammonium surfactant is preferably selected from the compounds with the general formula

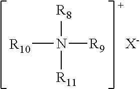

wherein R₈ is a saturated or unsaturated, branched or straight alkyl chain with 8-22 C atoms or

R₁₂—CO—NH—(CH₂)ₙ—

wherein R₁₂ is a saturated or unsaturated, branched or straight alkyl chain with 7-21 C atoms and n is an integer of 1-4, or

R₁₂—CO—O—(CH₂)ₙ—

wherein R₁₂ is a saturated or unsaturated, branched or straight alkyl chain with 7-21 C atoms and n is an integer of 1-4, and

R₉, R₁₀ and R₁₁ are independent from each other an alkyl group with 1 to 4 carbon atoms, hydroxyl alky chain with 1 to 4 carbon atoms, or ethoxy or propoxy group with a number of ethoxy or propoxy groups varying in the range of 1 to 4, and X is chloride, bromide, methosulfate or ethosulfate.

Suitable cationic surfactants are, for example, long-chain quaternary ammonium compounds which can be used alone or in admixture with one another, such as cetyl trimethyl ammonium chloride, myristyl trimethyl ammonium chloride, behentrimonium chloride, trimethyl cetyl ammonium bromide, stearyl trimethyl ammonium chloride, stearyl trimonium chloride and stearamidopropyltrimonium chloride.

In one embodiment, the quaternary ammonium salt comprising two alkyl chains is a non-cyclic quaternary ammonium salt of the following formula (II):

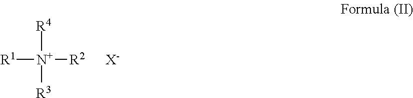

wherein R¹ and R² are selected from the group consisting of:
optionally substituted C₅-C₂₄ alkyl;
optionally substituted C₅-C₂₄ alkenyl;
optionally substituted C₅-C₂₄ alkynyl;
groups of the formula R′—CO—NH—(CH₂)ₙ—, wherein R′ is an optionally substituted C₅-C₂₄ alkyl, C₅-C₂₄ alkenyl or C₅-C₂₄ alkynyl group and n is an integer of 1 to 4; and
groups of the formula R′—CO—O—(CH₂)ₙ—, wherein R′ and n are the same as defined above;
   the optional substituent(s) being selected from halogen, hydroxyl, amino and C₁-C₄ alkoxy;
R³ and R⁴, which may be identical or different, represent an alkyl group with 1 to 4 carbon atoms, which may optionally be substituted with one or more hydroxyl groups or ethylene oxide and/or propylene oxide adducts thereof, the average addition number being in the range of 1 to 4; and
X⁻ represents an anion such as chloride, bromide, methosulfate or ethosulfate.

As preferable examples for such compounds of formula (II), di-C₁₂-C₁₅-alkyl dimethylammonium or di-C₁₂-C₁₅-alkyl hydroxyethylmonium methylsulfonates may be mentioned.

In case one or both of R¹ and R² represent a group of the formula R′—CO—NH—(CH₂)ₙ— or R′—CO—O—(CH₂)ₙ—, n is preferably 2 and the R′—CO-moiety is preferably derived from a saturated or unsaturated C₈-C₂₂-fatty acid or a mixture of such fatty acids. As a preferable example for such a compound, dioleylethyl hydroxyethylmonium methosulfate (TETRANYL® CO-40, commercially available from KAO CORPORATION) may be mentioned.

In another preferred embodiment, the quaternary ammonium salt is a cyclic compound of the imidazoline type, represented by the following formula (III):

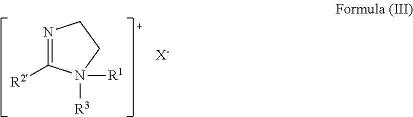

wherein R¹ and R³ are the same as for the formula (II) defined above, and e represents a linear or branched C₅-C₂₄ alkyl, C₅-C₂₄ alkenyl or C₅-C₂₄ alkynyl group which may be optionally substituted with at least one substituent selected from halogen, hydroxyl, amino and C₁-C₄ alkoxy.

As apparent from formula (III), these imidazoline type quats may be seen as the quaternized cyclization product of an acyl amide compound of the formula R^{2′}—CO—NR¹—CH₂—CH₂—NH₂, wherein the NH₂ group undergoes a cyclization with the carbonyl group, and the resulting imidazoline ring is quaternized with R³.

Preferably, R¹ in formula (III) represents a group of the formula R^{1′}—CO—NH—CH₂CH₂—, wherein the definition of R¹ is the same as R^{2′}. In this case, the compound of the formula (III) may be seen as the quaternized cyclization product of diacylated diethylene triamine. The acyl groups R^{1′}—CO— and R^{2′}—CO— are preferably selected from saturated or unsaturated fatty acids and mixtures thereof.

As a preferable example thereof, Quaternium-91 (di-behenyl imidazoline quat, methosulfate salt) may be mentioned. The commercially available product Crodazosoft™ DBP-Q (manufactured by Croda Inc.) is a mixture of Quaternium-91, cetrimonium methosulfate and cetearyl alcohol, and may be preferably used for the present invention.

In the composition of the present invention, it is possible to use only a single type of the quaternary ammonium salt having two C₅-C₂₄ linear or branched, saturated or unsaturated hydrocarbon groups in the molecule, or a combination of two or more types.

The content of the at least one quaternary ammonium salt, such as the quaternary ammonium salt of the formula (II) and/or (III), in the composition is usually 0.01 to 15 wt %, more preferably 0.05 to 10 wt. %, and even more preferably 0.1 to 5 wt. %, based on the total weight of the straightening composition.

Examples of the nonionic surfactant include polyoxy-C₁₋₄-alkylene C₈₋₂₄-alkyl ether, polyoxy-C₁₋₄-alkylene C₈₋₂₄-alkylene alkenyl ether, higher (C₁₂-C₂₄) fatty acid sucrose ester, polyglycerin C₈₋₂₄-fatty acid ester, higher (C₁₂-C₂₄) fatty acid mono- or diethanolamide, polyoxyethylene hardened castor oil, polyoxyethylene sorbitan C₈₋₂₄-fatty acid ester, polyoxyethylene sorbit C₈₋₂₄-fatty acid ester, C₈₋₂₄-alkyl saccharide surfactant, C₈₋₂₄-alkylamine oxide, and C₈₋₂₄-alkylamidoamine oxide.

Examples of the amphoteric surfactant include an imidazoline-based surfactant, a carbobetaine-based surfactant, an amidobetaine-based surfactant, a sulfobetaine-based surfactant, a hydroxysulfobetaine-based surfactant and an amidosulfobetaine-based surfactant.

Examples of the anionic surfactant include alkylbenzenesulfonate, alkyl or alkenyl ether sulfate, alkyl or alkenyl sulfate, olefin sulfonate, alkanesulfonate, saturated or unsaturated fatty acid salts, alkyl or alkenyl ether carboxylate, α-sulfo fatty acid salts, N-acylamino acid type surfactants, phosphoric acid mono- or diester type surfactants, and sulfosuccinate. Examples of the alkyl ether sulfate include polyoxyethylene alkyl ether sulfate. Examples of the counterion for the anionic residues of these surfactants include an alkalimetal ion such as sodium ion or potassium ion; an alkaline earth metal ion such as calcium ion or magnesium ion; an ammonium ion; and an alkanolamine having 1 to 3 alkanol groups each having 2 or 3 carbon atoms (for example, monoethanolamine, diethanolamine, triethanolamine, or triisopropanolamine).

The surfactant can be used singly or in combination of two or more kinds. When adding a surfactant to the treatment composition, the content thereof usually is 0.05 to 10% wt. %, more preferably 0.1 to 5 wt. %, based on the total weight of the treatment composition.

### 7. Conditioning Component

The treatment composition may optionally comprise a conditioning component suitable for application to the hair. The conditioning component is an oil or polymer which adheres to the hair and improves the feel and the manageability. When using the conditioning component, the total content thereof is preferably 0.01 to 30 wt. %, more preferably 0.05 to 20 wt. %, and even more preferably 0.1% to 10 wt. %, based on the total weight of the treatment composition.

Examples of the conditioning component generally include cationic polymers, silicones, higher alcohols, and organic conditioning oils (for example, hydrocarbon oil, polyolefin and fatty acid ester). The composition may comprise a single type of conditioning component, or two or more in combination.

### Cationic Polymers

A cationic polymer is a polymer having a cationic group or a group capable of being ionized into a cationic group, and in general, an amphoteric polymer acquiring net cationic charge is also included in the terminology. That is, the cationic polymer is a polymer containing an amino group or an ammonium group in a side chain of the polymer chain, or a polymer including a diallyl quaternary ammonium salt as a constituent unit, and examples thereof include cationized cellulose, cationic starch, cationic guar gum, a polymer or copolymer of a diallyl quaternary ammonium salt, and quaternized polyvinylpyrrolidone. Among these, from the viewpoint of softness, smoothness and easy finger-combing during shampooing, and easy manageability and moisture retention during drying, and from the viewpoint of stability of the agent, a polymer including a diallyl quaternary ammonium salt as a constituent unit, quaternized polyvinylpyrrolidone, and cationized cellulose are preferred, and a polymer or copolymer of a diallyl quaternary ammonium salt, and cationized cellulose are more preferred.

Specific examples of the polymer or copolymer of a diallyl quaternary ammonium salt include dimethyldiallylammonium chloride polymer (polyquaternium-6, for example, MERQUAT 100; Nalco Company), dimethyldiallylammonium chloride/acrylic acid copolymer (polyquaternium-22, for example, MERQUAT 280, MERQUAT 295; Nalco Company), and dimethyldiallylammonium chloride/acrylic acid amide copolymer (polyquaternium-7, for example, MERQUAT 550; Nalco Company).

Specific examples of the quaternized polyvinylpyrrolidone include quaternary ammonium salts synthesized from a copolymer of vinylpyrrolidone (VP) and dimethylaminoethyl methacrylate, and diethyl sulfate (polyquaternium 11, for example, GAFQUAT 734, GAFQUAT 755 and GAFQUAT 755N (all by ISP Japan, Ltd.)).

Specific examples of the cationized cellulose include a polymer of a quaternary ammonium salt obtained by adding glycidyltrimethylammonium chloride to hydroxyethylcellulose (polyquaternium-10, for example, RHEOGUARD G and RHEOGUARD GP (all by Lion Corp.), POLYMER JR-125, POLYMER JR-400, POLYMER JR-30M, POLYMER LR-400 and POLYMER LR-30M (all by Amerchol Corp.)), and a hydroxyethylcellulose/dimethyldiallylammonium chloride copolymer (polyquaternium-4, for example, CELQUAT H-100, CELQUAT L-200 (all by National Starch and Chemical Company)).

The cationic polymer may be used in combination of two or more kinds. Furthermore, the cationic polymer gives better effects when the content is increased, but an excessively high content of the cationic polymer may cause stability failure and a decrease in the viscosity of the agent alone or during mixing. From this viewpoint, and from the viewpoint of enhancing the feel to the touch, the content of the cationic polymer is preferably 0.001 to 20 wt %, more preferably 0.01 to 10 wt. %, and even more preferably 0.05 to 5 wt. %, based on the total weight of the treatment composition.

### Silicones

In order to improve the feel of use, the treatment composition preferably contains a silicone. Examples of the silicone include dimethylpolysiloxane, and modified silicone (for example, amino-modified silicone, fluorine-modified silicone, alcohol-modified silicone, polyether-modified silicone, epoxy-modified silicone, or alkyl-modified silicone), but dimethylpolysiloxane, polyether-modified silicone and amino-modified silicone are preferred.

The dimethylpolysiloxane may be any cyclic or non-cyclic dimethylsiloxane polymer, and examples thereof include SH200 series, BY22-019, BY22-020, BY11-026, B22-029, BY22-034, BY22-050A, BY22-055, BY22-060, BY22-083, FZ-4188 (all by Dow Corning Toray Co., Ltd.), KF-9008, KM-900 series, MK-15H, and MK-88 (all by Shin-Etsu Chemical Co., Ltd.).

The polyether-modified silicone may be any silicone having a polyoxyalkylene group, and the group constituting the polyoxyalkylene group may be an oxyethylene group or an oxypropylene group. More specific examples include KF-6015, KF-945A, KF-6005, KF-6009, KF-6013, KF-6019, KF-6029, KF-6017, KF-6043, KF-353A, KF-354A, KF-355A (all by Shin-Etsu Chemical Co., Ltd.), FZ-2404, SS-2805, FZ-2411, FZ-2412, SH3771M, SH3772M, SH3773M, SH3775M, SH3749, SS-280X series, BY22-008 M, BY11-030, and BY25-337 (all by Dow Corning Toray Co., Ltd.).

The amino-modified silicone may be any silicone having an amino group or an ammonium group, and examples thereof include an amino-modified silicone oil having all or a part of the terminal hydroxyl groups capped with a methyl group or the like, and an amodimethicone which does not have the terminals capped. A preferred example of the amino-modified silicone may be a compound represented by the following formula:

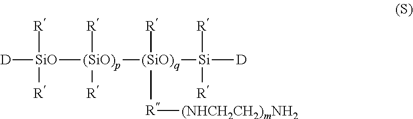

wherein R′ represents a hydroxyl group, a hydrogen atom or R^{x}; R^{x} represents a substituted or unsubstituted monovalent hydrocarbon group having 1 to 20 carbon atoms; D represents R^{x}, R″—(NHCH₂CH₂)ₘNH₂, OR^{x}, or a hydroxyl group; R″ represents a divalent hydrocarbon group having 1 to 8 carbon atoms; m represents a number from 0 to 3; p and q represent numbers, the sum of which is, as a number average, equal to or greater than 10 and less than 20,000, preferably equal to or greater than 20 and less than 3000, more preferably equal to or greater than 30 and less than 1000, and even more preferably equal to or greater than 40 and less than 800.

Specific examples of suitable commercially available products of the amino-modified silicone include amino-modified silicone oils such as SF8452C, SS-3551 (all by Dow Corning Toray Co., Ltd.), KF-8004, KF-867S, and KF-8015 (all by Shin-Etsu Chemical Co., Ltd.); and amodimethicone emulsions such as SM8704C, SM8904, BY22-079, FZ-4671, and FZ-4672 (all by Dow corning Toray Co., Ltd.).

The total content of these silicones in the treatment composition of the present invention is usually 0.1 to 20 wt. %, preferably 0.2% to 10 wt. % and more preferably 0.5 to 5 wt. %, based on the total weight of the treatment composition.

### Oil Component

For improving the feel upon use, the treatment composition may also include an organic conditioning oil. The organic conditioning oil that is suitably used as a conditioning component is preferably a low-viscosity and water-insoluble liquid, and is selected from a hydrocarbon oil having at least 10 carbon atoms, a polyolefin, a fatty acid ester, a fatty acid amide, a polyalkylene glycol, and mixtures thereof. The viscosity of such an organic conditioning oil as measured at 40° C. is preferably 1 to 200 mPa·s, more preferably 1 to 100 mPa·s, and even more preferably 2 to 50 mPa·s. For the determination of the viscosity, a capillary viscometer may be used.

Examples of the hydrocarbon oil include a cyclic hydrocarbon, a linear aliphatic hydrocarbon (saturated or unsaturated), and a branched aliphatic hydrocarbon (saturated or unsaturated), and polymers or mixtures thereof are also included. The linear hydrocarbon oil preferably has 12 to 19 carbon atoms. The branched hydrocarbon oil includes hydrocarbon polymers, and preferably has more than 19 carbon atoms.

The polyolefin is a liquid polyolefin, more preferably a liquid poly-α-olefin, and even more preferably a hydrogenated liquid poly-α-olefin. The polyolefin used herein is prepared by polymerizing an olefin monomer having 4 to 14 carbon atoms, and preferably 6 to 12 carbon atoms.

The fatty acid ester may be, for example, a fatty acid ester having at least 10 carbon atoms. Examples of such a fatty acid ester include esters having a hydrocarbon chain derived from a fatty acid and an alcohol (for example, monoesters, polyhydric alcohol esters, or di- and tricarboxylic acid esters). The hydrocarbon group of these fatty acid esters may have another compatible functional group such as an amide group or an alkoxy group as a substituent, or the hydrocarbon group may be covalently bonded to those functional groups. More specifically, an alkyl and alkenyl ester of a fatty acid having a fatty acid chain having 10 to 22 carbon atoms, a carboxylic acid ester of an aliphatic alcohol having an aliphatic chain derived from an alkyl and/or alkenyl alcohol having 10 to 22 carbon atoms, and a mixture thereof are suitably used. Specific examples of these preferred fatty acid esters include isopropyl isostearate, hexyl laurate, isohexyl laurate, isohexyl palmitate, isopropyl palmitate, decyl oleate, isodecyl oleate, hexadecyl stearate, decyl stearate, dihexadecyl adipate, lauryl lactate, myristyl lactate, cetyl lactate, oleyl stearate, oleyl oleate, oleyl myristate, lauryl acetate, cetyl propionate and dioleyl adipate.

Further suitable oil components are natural oils such as paraffin oil and natural triglycerides.

Suitable natural triglycerides are argan oil, shea butter oil, karite oil, olive oil, almond oil, avocado oil, ricinus oil, coconut oil, palm oil, sesame oil, peanut oil, sunflower oil, peach kernel oil, wheat germ oil, macadamia nut oil, macadamia oil, night primrose oil, jojoba oil, castor oil, soya oil, lanolin, passiflora oil, black cumin oil, borage oils, grapeseed oil, hempseed oil, kukui nut oil, and rosehip oil.

The organic conditioning oil may be used in combination of two or more kinds, and the total concentration is typically in the range of 0.1 to 20 wt. %, preferably 0.2 to 10 wt. %, more preferably 0.5 to 5 wt. %, based on the total weight of the treatment composition.

### Fatty Alcohols

From the viewpoint of improving the sense of touch and stability, the treatment composition may also contain a higher alcohol having 8 carbon atoms or more. Usually, the higher alcohol has 8 to 22 carbon atoms, and preferably 16 to 22 carbon atoms. Specific examples thereof include cetyl alcohol, stearyl alcohol, behenyl alcohol, and mixtures thereof.

The higher alcohol may be used in combination of two or more kinds, and the content thereof is typically 0.1 to 20 wt. %, preferably 0.2 to 10 wt. %, more preferably 0.5 to 5 wt. %, based on the total weight of the treatment composition.

Additionally polyols may suitably be comprised in the compositions. Examples of the polyalkylene glycol include polyethylene glycol and polypropylene glycol, and a mixture of the two may be used, or a copolymer of ethylene oxide and propylene oxide may also be used.

### 8. Thickening Polymer

The treatment composition, specifically the dyeing/straightening composition and the straightening composition, may further comprise a thickening polymer. Polymeric thickening agent is preferably selected from non-ionic, anionic, cationic and amphoteric polymers having a viscosity of at least 500 mPa·s, preferably 1,000 mPa·s, more preferably 1,500 mPa·s most preferably 2,000 mPa·s measured at a polymer concentration of 1% by weight in water and at 20° C. with a Brookfield viscometer, such as at 10 rpm for 1 minute with an appropriate spindle.

The polymeric thickening agent is selected from non-ionic, anionic, cationic and amphoteric polymers having a viscosity of at least 500 mPa·s measured at a polymer concentration of 1% by weight in water and at 20° C. with a Brookfield viscometer, such as at 10 rpm for 1 minute, with an appropriate spindle.

The total concentration of the one or more thickening agent is typically within the range of 0.01 to 15 wt. %, preferably 0.05 to 10 wt. %, more preferable 0.1 to 5 wt. %, and most preferably 0.5 to 2 wt. % based on the weight of the straightening composition.

### Non-Ionic Polymers

Any cosmetically acceptable non-ionic thickening polymers, which are usually employed in the field of hair cosmetic compositions, may be used as the polymeric thickening agent in the composition of the present invention. The composition may include a single kind or multiple kinds of non-ionic polymer, and may further include one or more anionic, cationic and/or amphoteric polymers in combination with the non-ionic polymer.

Examples for the non-ionic polymers include neutral polysaccharides and derivatives such as ethers or esters thereof. In this respect, neutral gums such as guar gum, hydroxypropyl guar, hydroxyethylcellulose (HEC), methyl hydroxyethylcellulose (MHEC), ethyl hydroxyethylcellulose (EHEC), methyl ethyl hydroxyethylcellulose (MEHEC), hydroxypropylcellulose (HPC), hydroxypropylmethylcellulose (HPMC), hydrophobically modified derivatives thereof such as HM-EHEC, starch and dextrins may be mentioned.

Cellulose ethers such as HEC, MHEC or MEHEC are preferable. As commercially available examples, STRUCTURE® CEL 12000 M (MEHEC)[STRUCTURE® CEL 4400 E (EHEC) and STRUCTURE® CEL 500 M (HM-EHEC) may be mentioned (available from AkzoNobel, the number indicating the viscosity in cps).

### Anionic Polymers

Any cosmetically acceptable anionic thickening polymers, which are usually employed in the field of hair cosmetic compositions, may be used as the polymeric thickening agent in the composition of the present invention. The composition may include a single kind or multiple kinds of anionic polymer, and may further include one or more non-ionic, cationic and/or amphoteric polymers in combination with the anionic polymer.

Examples for the anionic polymer include anionic polysaccharides and derivatives thereof, such as alginate, pectin, hyaluronate, anionic gums such as xanthan gum, dehydroxanthan gum, hydroxypropyl xanthan gum, gum arabic, gum karaya or gum tragacanth, or anionic cellulose derivatives such as carboxymethyl cellulose (CMC).

Further examples include synthetic anionic polymers such as polyacrylic acid or copolymers containing acrylic acid in combination with neutral vinylic and/or acrylic monomers, and salts thereof such as sodium polyacrylate.

Xanthan gum, hydroxypropyl xanthan gum and dehydroxanthan gum are particularly preferable in view of pH stability.

### Cationic Polymers

Any cosmetically acceptable cationic thickening polymers, which are usually employed in the field of hair cosmetic compositions, may be used as the polymeric thickening agent in the composition of the present invention. The composition may include a single kind or multiple kinds of cationic polymer, and may further include one or more non-ionic, anionic and/or amphoteric polymers in combination with the cationic polymer.

A cationic polymer is a polymer having a cationic group or a group capable of being ionized into a cationic group. The cationic polymer typically is a polymer containing an amino group or an ammonium group in a side chain of the polymer chain, or a polymer including a diallyl quaternary ammonium salt as a constituent unit, and examples thereof include cationized cellulose, cationic starch, cationic guar gum, a vinylic or (meth)acrylic polymer or copolymer having quaternary ammonium side chains, a polymer or copolymer of a diallyl quaternary ammonium salt, and quaternized polyvinylpyrrolidone.

As an example of the of the vinylic or (meth)acrylic polymer or copolymer having quaternary ammonium side chains, poly(2-methacryloxyethyltrimethylammonium chloride) (Polyquaternium-37) may be mentioned.

Specific examples of the cationized cellulose include a polymer of a quaternary ammonium salt obtained by adding glycidyltrimethylammonium chloride to hydroxyethylcellulose (polyquaternium-10, for example, RHEOGUARD G and RHEOGUARD GP (all by Lion Corp.), POLYMER JR-125, POLYMER JR-400, POLYMER JR-30M, POLYMER LR-400 and POLYMER LR-30M (all by Amerchol Corp.)), a hydroxyethylcellulose/dimethyldiallylammonium chloride copolymer (polyquaternium-4, for example, CELQUAT H-100, CELQUAT L-200 (all by National Starch and Chemical Company)), and a polymeric quaternary ammonium salt of hydroxyethyl cellulose reacted with a trimethyl ammonium substituted epoxide and a lauryl dimethyl ammonium substituted epoxide (polyqauternium-67, for example, SoftCat Polymer SX-1300H).

Among these examples, polyquaternium-37 is particularly preferable.

### Amphoteric Polymers

The amphoteric polymer contains both, cationic and anionic groups. From a structural point of view, the amphoteric polymer may be derived from any of the described cationic polymer types by the additional introduction of anionic groups or co-monomers.

Like the cationic polymers, the amphoteric polymers may also provide a hair conditioning effect, specifically in case of a positive net charge.

Any cosmetically acceptable amphoteric thickening polymers, which are usually employed in the field of hair cosmetic compositions, may be used as the polymeric thickening agent in the composition of the present invention. The composition may include a single kind or multiple kinds of amphoteric polymer, and may further include one or more non-ionic, anionic and/or cationic polymers in combination with the amphoteric polymer.

As examples for the amphoteric polymer, carboxyl-modified or sulfonate-modified cationic polysaccharides such as carboxymethylchitosan may be mentioned.

Further examples include copolymers of cationic vinylic or (meth)acrylic monomers with (meth)acrylic acid, such as dimethyldiallylammonium chloride/acrylic acid copolymer (polyquaternium-22, for example, MERQUAT 280, MERQUAT 295; Nalco Company).

As an amphoteric polymer, polyquaternium-22 is particularly preferable.

### 9. Hair Treatment Processes

The steps of the embodiments of the first to sixth aspect will now be described in further detail.

An embodiment of the first aspect of the present invention comprises the steps (i-a) to (i-f) as defined above. In step (i-a), the straightening/dyeing composition is applied to the hair. The application weight ratio of hair to composition is 0.5:2 to 2:0.5, preferably 0.5:1 to 1:0.5, more preferably about 1:1.

Subsequent to the application, the treatment composition is left on the hair for 1 to 120 minutes, preferably 5 to 90 minutes, more preferably 10 to 60 minutes and most preferably 15 to 45 minutes at a temperature of 45° C. or below, preferably at ambient temperature (step (i-b)). Then, the straightening composition is optionally rinsed off from the hair (step (i-c)).

In subsequent step (i-d), the hair is dried in order to avoid an excessive steam generation in the subsequent step of treating the hair with the iron. Typically, a hair dryer is used for this purpose. It is preferable to dry the hair under continuous combing in order to prevent entanglement of the hair.

Subsequent to the drying, the hair is treated with an iron having a surface temperature of 130 to 250° C., more preferably 150 to 230° C., even more preferably 170 to 200° C. A usual straightening iron may be used for this purpose (step (i-e)). Finally, the hair may be optionally rinsed off and/or shampooed and dried (step (i-f)).

An embodiment of the second aspect of the present invention generally involves two stages, namely a coloring treatment (e.g., steps (ii-a) to (ii-c) as defined above) and a straightening treatment (e.g., steps (ii-d) to (ii-i)).

In the coloring treatment, the dye composition is applied to the hair (step (ii-a)) and left on the hair for 5 to 45 minutes, preferably 5 to 30 minutes, and is rinsed off with water(step (ii-b)). Optionally, the hair may be shampooed.

The application ratio of hair to composition by weight is preferably 0.5:2 to 2:0.5, more preferably 0.5:1 to 1:0.5, even more preferably about 1:1. Optionally, the hair may be dried after the coloring step, in order to reduce the water content of the hair. A hair dryer may be used for this purpose (step (ii-c)).

After completion of the coloring treatment (steps (ii-a) to (ii-c)), the hair is subjected to the straightening treatment in accordance with steps (ii-d) to (ii-i). These steps may be performed under essentially the same conditions as described above for the simultaneous dyeing/straightening treatment of the first aspect of the invention, and carried out directly after the coloring treatment. In this connection, “directly after” means that the time interval between the completion of steps (ii-a) to (ii-c) and the beginning of steps (ii-d) to (ii-i) is not more than two or three hours, preferably not more than 1 hour, more preferably not more than 30 minutes.

An embodiment of the third aspect of the present invention comprises the steps (iii-a) to (iii-f) as defined above, and differs from the above-discussed embodiment of the second aspect in that the order of dyeing and straightening treatment is reversed. The straightening treatment and the dyeing treatment may be performed under the same conditions as described above for the second aspect.

Embodiments of the fourth and fifth aspects of the invention comprise the above-defined steps (iv-a) to (iv-g) and (v-a) to (v-g), respectively. Steps (iv-a) to (iv-c) relate to a bleaching treatment.

In step (iv-a), a bleaching composition is applied to the hair and allowed to act for 5 to 45 minutes before it is rinsed off (step (iv-b)). In order to achieve an even bleaching or lightening, the time preferably is 5 minutes or longer, more preferably 10 minutes or longer. In general, a maximum time of 30 minutes is usually sufficient. The application weight ratio of hair to composition is preferably from 0.5:2 to 2:0.5, preferably 0.5:1 to 1:0.5, more preferably about 1:1. Then, the composition is rinsed off with water. Optionally, the hair may be shampooed after the rinsing and dried, e.g., by using a hair dryer (step (iv-c)).

Directly after completion of the bleaching treatment, the hair is subjected to the straightening treatment in accordance with steps (iv-d) to (iv-g). These steps may be performed under the same conditions as the respective straightening steps of the second aspect of the invention.

The Fifth aspect differs from the fourth aspect in the reversed order of the treatments. The straightening treatment and the bleaching treatment in the fifth aspect may be performed under the same conditions as in the fourth aspect.

An embodiment of the sixth aspect involves the above-defined steps (vi-a) to (vi-e). In steps (vi-a) to (vi-c), the straightening composition is applied to the hair at a ratio of hair to composition by weight is preferably 0.5:2 to 2:0.5, more preferably 0.5:1 to 1:0.5, even more preferably about 1:1. Then, the composition is left on the hair for 1 to 45 minutes and optionally rinsed off.

According the sixth aspect of the present invention, successful hair straightening and frizz reduction is also achieved when hair is dried under mechanical tension and hot air having a temperature in the range of 60-140° C. (step (vi-d)), instead of the treatment with a straightening iron. The temperature refers to the temperature at the hair.

Drying under conditions of mechanical tension means that mechanical tension is applied either along the whole length of the strand of hair to be dried, or along the part of the strand exposed to the hot air stream.

The mechanical tension during the drying may be applied to the hair continuously by holding a part or the whole of the strand in an elongated state while applying the hot air flow to the strand or the elongated part thereof. It is also possible to apply the tension intermittently, e.g., by continuous combing of the strand with a comb while applying the hot air stream.

### EXAMPLES

The present invention is now illustrated by the following non-limiting examples.

### Example 1

An aqueous hair dyeing/straightening composition comprising anionic dyes is formulated by mixing the following ingredients:

| 10.00 | wt. % | Formaldehyde |
|---|---|---|
| 1.50 | wt. % | Amodimethicone |
| 1.40 | wt. % | Hydroxyethylcellulose |
| 0.30 | wt. % | Fragrance |
| 0.55 | wt. % | Acid Red 52/C.I. 45100 |
| 0.28 | wt. % | Orange 4/Acid Orange 7/C.I. 15510 |
| 0.01 | wt. % | Ext. Violet 2/Acid Violet 43/C.I. |
| q.s. to pH 3.0 | | Lactic acid |
| to 100 | wt. % | Water |

### Example 2

An aqueous dyeing/straightening composition comprising cationic dyes is formulated by mixing the following ingredients:

| 10.00 | wt. % | Formaldehyde |
|---|---|---|
| 1.50 | wt. % | Amodimethicone |
| 1.40 | wt. % | Hydroxyethylcellulose |
| 0.30 | wt. % | Fragrance |
| 0.55 | wt. % | Basic red 51 |
| 0.28 | wt. % | Basic yellow 87 |
| 0.01 | wt. % | Basic orange 31 |
| q.s.to pH 4.5 | | Lactic acid |
| to 100 | wt. % | Water |

### Example 3

An aqueous dyeing/straightening composition comprising nitro dyes is formulated by mixing the following ingredients:

| 10.00 | wt. % | Formaldehyde |
|---|---|---|
| 1.50 | wt. % | Amodimethicone |
| 1.40 | wt. % | Hydroxyethylcellulose |
| 0.30 | wt. % | Fragrance/Parfum |
| 0.55 | wt. % | HC Red 3 |
| 0.28 | wt. % | HC Yellow 2 |
| q.s. to pH 5.0 | | Lactic acid |
| to 100 | wt. % | Water |

### Example 4

An aqueous dyeing/straightening composition comprising a combination of an anionic dye, a cationic dye and a nitro dye is prepared by mixing the following ingredients:

| 10.00 | wt. % | Formaldehyde |
|---|---|---|
| 1.50 | wt. % | Amodimethicone |
| 1.40 | wt. % | Hydroxyethylcellulose |
| 0.30 | wt. % | Fragrance/Parfum |
| 0.55 | wt. % | Basic red 51 |
| 0.28 | wt. % | Acid red 52 |
| 0.010 | wt. % | HC red 3 |
| q.s. to pH 3.0 | | Lactic acid |
| to 100 | wt. % | Water |

### Example 5

### Straightening Composition

An aqueous straightening composition for use in the methods of the second to sixth aspect of the present invention is prepared by mixing the following ingredients:

| 10.00 | wt. % | Formaldehyde |
|---|---|---|
| 1.50 | wt. % | Amodimethicone |
| 1.40 | wt. % | Hydroxyethylcellulose |
| 0.30 | wt. % | Fragrance/Parfum |
| q.s. to pH 3.0 | | Lactic acid |
| to 100 | wt. % | Water |

### Example 6

### Dye Compositions

Dye compositions for use in the second and third aspect of the present invention are prepared by mixing the following ingredients.

% w/w

Direct Dye Composition

| | |
|---|---|
| 1.50 | Amodimethicone |
| 1.40 | Hydroxyethylcellulose |
| 0.30 | Fragrance/Parfum |
| 0.55 | Acid Red 52/C.I. 45100 |
| 0.28 | Basic red 51 |
| 0.01 | HC red 3 |
| To 100 | Water |

Oxidative Hair Dye
Composition for permanent colour

| | |
|---|---|
| 0.25 | Ammonium chloride |
| 8.00 | Ammonia 25% |
| 0.30 | Fragrance/Parfum |
| 0.55 | p-toluenediamine |
| 0.28 | resorcinol |
| 0.01 | m-phenylenediamine |
| To 100 | Water |
| pH: 9.5 | |

The permanent oxidative hair dye composition is mixed with a 6% H₂O₂ solution at a weight ratio of 1:1 before application to the hair.

Demi-permanent Dye Composition

| % w/w | |
|---|---|
| 0.25 | Ammonium chloride |
| 0.80 | Monoethanolamine |
| 0.30 | Fragrance/Parfum |
| 0.55 | p-toluenediamine |
| 0.28 | resorcinol |
| 0.01 | m-phenylenediamine |
| To 100 | Water |
| pH: 6.8 | |

The demi-permanent oxidative hair dye composition is mixed with a 6% H₂O₂ solution at a weight ratio of 1:1 before application to the hair.

### Example 7

### Bleaching Compositions

Bleach compositions for use in the fourth and fifth aspect of the present invention are prepared by mixing the following ingredients.

% by weight

Lightening composition

| | |
|---|---|
| H₂O₂ | 4% |
| Ammonia | 4% |
| Water | to 100 |

Bleach powder

| | |
|---|---|
| Sodium persulphate | 20 |
| Potassium persulphate | 25 |
| Sodium silicate | 10 |
| Paraffin oil | 10 |
| Diatomeous earth | to 100 |

Prior to application to the hair, the bleach powder is mixed with an aqueous 9% H₂O₂ solution at a weight ratio of 1:1.

1. A process for treating hair, characterized in that it comprises the following steps performed in this order:
(a) application of a dyeing/straightening composition comprising at least one direct dye and formaldehyde onto the hair;
(b) leaving the composition on the hair for 1 to 120 minutes;
(c) optionally rinsing off the hair;
(d) drying the hair; and
(e) treating the hair with an iron having a surface temperature of 180±50° C.
(f) optionally rinsing off and/or shampooing the hair and drying.

2. The process according to claim 1, wherein the dyeing/straightening composition comprises formaldehyde at a concentration in the range of 0.1 to 20% by weight in terms of HCHO, based on the weight of the total composition.

3. The process according to claim 1, wherein the dyeing/straightening composition is free of any sulfur-based reducing agents.

4. The process according to claim 1, wherein the direct dye is an anionic direct dye.

5. The process according to claim 1, wherein the direct dye is a cationic direct dye.

6. The process according to claim 1, wherein the direct dye is a nitro dye.

7. The process according to claim 1, wherein the total content of (the) direct dye(s) is 0.001 to 10 wt. %, based on the total composition.

8. The process according to claim 1, wherein the dyeing/straightening composition further comprises a silicone and/or a cationic polymer.

9. The process according to claim 1, wherein the dyeing/straightening composition is applied to the hair in an application weight ratio of hair to composition of 0.5:2 to 2:0.5.

10. The process according to claim 1, wherein the temperature of the iron is in the range of 170 to 200° C.

11. The process according to claim 1, wherein the dyeing/straightening composition has a pH in the range of 1 to 6.

12. A dyeing/straightening composition for the hair, comprising at least one direct dye and formaldehyde.

13. The dyeing/straightening composition according to claim 12, wherein the direct dye is anionic and/or cationic and/or nitro dye and, preferably the composition has a pH 1n the range of 1 to 6.

14. Kit for dyeing and straightening the hair, comprising a composition according to claim 12 and a straightening iron.
